(19) United States
(12) Patent Application Publication
(10) Pub. No.: US 2020/0260993 A1
(43) Pub. Date: Aug. 20, 2020

(54) METHOD AND APPARATUS FOR CLASSIFYING POSITION OF TORSO AND LIMB OF A MAMMAL

(71) Applicant: DORSAVI LTD, Victoria (AU)

(72) Inventors: Andrew James Ronchi, Victoria (AU); Meagan Simone Blackburn, Victoria (AU); Sangeeth Anuradha Wanasinghage, Victoria (AU); Sarah Patricia Elliott, Victoria (AU); Edgar Charry, Victoria (AU)

(21) Appl. No.: 16/648,164

(22) PCT Filed: Sep. 18, 2018
(86) PCT No.: PCT/AU2018/051020

§ 371 Date: Mar. 17, 2020

(30) Foreign Application Priority Data
Sep. 18, 2017 (AU) 2017903794

(51) Int. Cl. A61B 5/107; A61B 5/11; A61B 5/00

(52) U.S. Cl. CPC A61B 5/1071; A61B 5/1121; A61B 5/1116; A61B 5/1126; A61B 2503/40; A61B 5/7264; A61B 5/725; A61B 2505/09; A61B 2562/0219; A61B 5/4561

(57) ABSTRACT

An apparatus for providing a classification of an angular position of a limb relative to a torso of a vertebral mammal is provided. The apparatus includes a first sensor, a second sensor, a memory device and a processor. The first sensor is for measuring position of the torso relative to a first frame of reference and for providing first data indicative of the torso position. The second sensor is for measuring position of the limb relative to a second frame of reference and for providing second data indicative of the limb position. The memory device is adapted for storing the first and second data at least temporarily. The processor is adapted for processing the first and second data to derive the angular position of the limb relative to the torso in at least one anatomical plane of the mammal's body, and to provide the classification based at least on the derived torso and limb positions. A method for providing a classification of an angular position of a limb relative to a torso of a vertebral mammal is also provided.

This application claims priority from Australian Provisional Patent Application No. 2017903794 filed on 18 Sep. 2017, the contents of which are to be taken as incorporated herein by this reference.

### TECHNICAL FIELD

The present invention relates to an apparatus and method for ascertaining and classifying position of a torso and a limb of a vertebral mammal. It relates more particularly but not exclusively to the use of wearable sensors to measure and classify position of a spine and an upper limb of the mammal, or a pelvis and a lower limb of the mammal.

### BACKGROUND OF INVENTION

As stated by Dargel¹ in 2014, the rate of dislocation of primary hip replacements ranges from 0.2% to 10% per year, while that of revision surgery for artificial hip joints is as high as 28%. This is a significant figure when Dargel¹ states that the number of total hip replacements (THR) is expected to increase internationally by 170% by 2030.

The procedure-specific risk factors for THR dislocation can be divided into four categories: the surgical approach; positioning of the acetabular and femoral components; soft-tissue tension, and the surgeon's experience. In relation to positioning of the acetabular and femoral components, the alignment of the implants during hip replacement surgery is critical for the stability of the artificial joint. Although there is an endeavour to position the acetabular and femoral cup by individual anatomic requirements, the dislocation-stable cup position is with an inclination of 40±10° and an anteversion of 10 to 20°, as published by Lewinnek², is internationally considered standard.

Wines et al. in a 2006 study³ asked surgeons during an operation to estimate the alignment of the acetabular and femoral components and compared these estimations with postoperative computed-tomography (CT) scan measurements. It was found that when surgeons estimated intraoperatively an acetabular component anteversion between 10° and 30°, only 45% of components actually were within this target range. In the case of the femoral component alignment, the surgeons intraoperatively estimated the antetorsion in 93% of cases between 15° and 20°, while CT scan measurements ranged from 15° retrotorsion to 45° antetorsion and 71% of prosthesis stems were in the target range. While a component position which increases the risk of THA dislocation is a procedure-related factor and can potentially be avoided, it is influenced by intraoperative positioning, the patient-specific anatomical situation, periarticular contractures, malpositioning of the lumbosacral junction, and obesity as well as considerably by the surgeon's experience.

Similar observations can be considered for shoulder replacement surgery, which involves insertion and positioning of prosthetic components, namely the head of the humerus bone (ball) or replacement of both the ball and the glenoid (socket). Proper alignment of the implants during shoulder replacement surgery is critical for stability of the artificial joint. The intraoperative alignment of the implants relies on a number of factors, such as the surgeon's experience and the patient-specific anatomy, to name a few. Poor alignment of the prosthetic components increases the risk of shoulder dislocations post-surgery⁵.

There is a need to understand and accurately measure the static and dynamic positions of torso and lower or upper limb in the lead up to, during and following hip and shoulder replacement surgery. Due to the inaccuracies of clinical assessments, costs, radiation levels and the complexity of current radiographic and/or magnetic resonance (MR) techniques, the ability to use wearable sensors to measure these positions is attractive.

Therefore, it would be desirable to provide an apparatus and a method that involves the use of wearable sensors to measure position of the torso and lower or upper limb, and which ameliorate and/or overcome one or more problems and/or inconveniences of the prior art.

A reference herein to a patent document or any other matter identified as prior art, is not to be taken as an admission that the document or other matter was known or that the information it contains was part of the common general knowledge as at the priority date of any of the claims.

### SUMMARY OF THE INVENTION

According to one aspect, the present invention provides an apparatus for providing classification of position of a torso and a limb of a body of a vertebral mammal. The apparatus includes: a first sensor for measuring position of the torso relative to a first frame of reference and for providing first data indicative of the torso position; a second sensor for measuring position of the limb relative to a second frame of reference and for providing second data indicative of the limb position; a memory device adapted for storing the first and second data at least temporarily; and a processor adapted for processing the first and second data to derive angular positions of the torso and the limb in at least one anatomical plane of the mammal's body, and to provide the classification based at least on the derived torso and limb positions.

In some embodiments, the processor is further adapted to execute a position algorithm for deriving an angular position of the limb relative to the torso in at least one anatomical plane of the mammal's body. The position algorithm may be adapted to transform the second data from the second frame of reference relative to the first frame of reference to derive the angular position of the limb relative to the torso.

In some embodiments, the processor is further adapted to execute a classification algorithm for providing the classification based on at least on the derived torso and limb positions. The classification may also be based on other personal data, such as BMI, age, weight, height or other attributes relevant to the assessment.

The processor may be further adapted to receive reference data, and the classification algorithm may include a comparator adapted to compare the derived torso and limb positions to the reference data. The reference data may include one or more threshold values or a range of values for the torso and limb positions based on a normative population of vertebral mammals.

In some embodiments, the classification algorithm includes a classifier adapted to classify the derived torso and limb positions based on the comparison by the comparator. The classifier may be adapted to classify the derived torso and limb positions when the derived torso and limb positions are more than or less than the threshold values or fall within the range of values of the torso and limb positions from the reference data.

The classifier may be adapted to classify the derived torso position as at least one of: anterior, neutral or posterior in a sagittal plane of the mammal's body; left, neutral or right in a coronal plane of the mammal's body; and left, neutral or right rotation in a transverse plane of the mammal's body. The classifier may also be adapted to classify the derived limb position as at least one of: flexion, neutral or extension in a sagittal plane of the mammal's body; abduction, neutral or adduction in a coronal plane of the mammal's body; and internal, neutral or external rotation in a transverse plane of the mammal's body.

The classifier may be adapted to at least classify the derived torso position in a sagittal plane of the mammal's body and classify the derived limb position in a transverse plane of the mammal's body.

In some embodiments, the classification algorithm is further adapted to assign a class signature based on the classification of the derived torso and limb positions. The class signature may be indicative of stability of a hip and/or a shoulder joint of the mammal. The processor may be further adapted to generate an automated report on the classification, where the automated report includes one or both of the classified torso and limb positions and the class signature.

The classification may be provided in static and/or dynamic states of the torso and limb. The processing may also be performed in real-time to provide feedback on the classification to a user. For example, the user may include the mammal or an operator of the apparatus, including a medical practitioner or surgeon.

In some embodiments, one or both of the first sensor and the second sensor includes at least one acceleration sensor adapted for measuring acceleration along one or more orthogonal axes. One or both of the first sensor and the second sensor may include at least one rotation sensor adapted for measuring rotation around one or more orthogonal axes. The first and second sensors may include at least one of an accelerometer, a gyroscope and a magnetometer.

Additionally, the first and second sensors may include at least one analog to digital (A to D) converter for converting analog data to a digital domain, and the A to D converter may be adapted to convert an analog output from the first and second sensors to the first and second data, respectively, prior to storing the first and second data.

In some embodiments, the apparatus further includes a third sensor for measuring muscle activity of the torso and/or limb of the mammal, and for providing third data indicative of the muscle activity.

The torso may include a pelvis or a spine of the mammal. Where the torso includes the pelvis, the limb may include a lower limb of the mammal. The lower limb may include a femur or a tibia of the mammal, and preferably includes the femur. Where the torso includes the spine, the limb may include an upper limb of the mammal. The upper limb may include a radius, an ulna or a humerus of the mammal, and preferably include the humerus.

According to another aspect, the present invention provides a method for providing classification of position of a torso and a limb of a body of a vertebral mammal. The method includes: measuring position of the torso relative to a first frame of reference using a first sensor; measuring position of the limb relative to a second frame of reference using a second sensor; providing first data indicative of the torso position and second data indicative of the limb position; storing the first and second data at least temporarily; and processing the first and second data to derive angular positions of the torso and the limb in at least one anatomical plane of the mammal's body, and to provide the classification based at least on the derived torso and limb positions.

In some embodiments, the processing further includes performing a position algorithm for deriving an angular position of the limb relative to the torso in at least one anatomical plane of the mammal's body. Performing the position algorithm may include transforming the second data from the second frame of reference relative to the first frame of reference to derive the angular position of the limb relative to the torso.

In some embodiments, the processing further includes performing a classification algorithm for providing the classification based at least on the derived torso and limb positions.

The method may further include receiving reference data, and performing the classification algorithm may include comparing, using a comparator of the classification algorithm, the derived torso and limb positions to the reference data. The reference data may include one or more threshold values or a range of values for the torso and limb positions based on a normative population of vertebral mammals.

In some embodiments, performing the classification algorithm further includes classifying, using a classifier of the classification algorithm, the derived torso and limb positions based on the comparison by the comparator. The classifying may include classifying the derived torso and limb positions when the derived torso and limb positions are more than or less than the threshold values or fall within the range of values of the torso and limb positions from the reference data.

In some embodiments, the derived torso position is classified as at least one of: anterior, neutral or posterior in a sagittal plane of the mammal's body; left, neutral or right in a coronal plane of the mammal's body; and left, neutral or right rotation in a transverse plane of the mammal's body. In some embodiments, the derived limb position is classified as at least one of: flexion, neutral or extension in a sagittal plane of the mammal's body; abduction, neutral or adduction in a coronal plane of the mammal's body; and internal, neutral or external rotation in a transverse plane of the mammal's body.

The classifying may include at least classifying the derived torso position in a sagittal plane of the mammal's body and classifying the derived limb position in a transverse plane of the mammal's body.

In some embodiments, performing the classification algorithm further includes assigning a class signature based on the classification of the derived torso and limb positions. The class signature may be indicative of stability of a hip and/or a shoulder joint of the mammal. The method may further include generating an automated report on the classification, where the automated report includes one or both of the classified torso and limb positions and the class signature.

The classification may be provided in static and/or dynamic states of the torso and limb. The processing may also be performed in real-time to provide feedback on the classification to a user. For example, the user may be the mammal or an operator, including a medical practitioner or a surgeon.

In some embodiments, one or both of the first sensor and the second sensor includes at least one acceleration sensor adapted for measuring acceleration along one or more orthogonal axes. One or both of the first sensor and the second sensor may include at least one rotation sensor adapted for measuring rotation around one or more orthogonal axes. The first and second sensors may include at least one of an accelerometer, a gyroscope and a magnetometer.

Additionally, each step of measuring may include converting analog data to a digital domain using at least one analog to digital (A to D) converter of the first and second sensor, and where the A to D conversion takes place prior to storing the first and second data.

In some embodiments, the method further includes measuring muscle activity of the torso and/or limb of the mammal using a third sensor, and providing third data indicative of the muscle activity.

The torso may include a pelvis or a spine of the mammal. Where the torso includes the pelvis, the limb may include a lower limb of the mammal. The lower limb may include a femur or a tibia of the mammal, and preferably includes the femur. Where the torso includes the spine, the limb may include an upper limb of the mammal. The upper limb may include a radius, an ulna or a humerus of the mammal, and preferably include the humerus.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the claims appended hereto.

FIG. 1 shows an apparatus for providing classification of position of a torso and a limb of a body of a vertebral mammal according to an embodiment of the present invention;

FIG. 2 shows a flow chart illustrating processing of sensor data by a position algorithm according to an embodiment of the present invention;

FIG. 3 shows a flow chart illustrating processing of position data by a classification algorithm according to an embodiment of the present invention;

FIG. 4 shows a more detailed flow chart of the position algorithm of FIG. 2 according to an embodiment of the present invention;

FIG. 5 shows a flow chart illustrating steps in a method for providing classification of position of a torso and a limb of a body of a vertebral mammal according to an embodiment of the present invention;

FIG. 6 shows use of the inventive apparatus and method in conjunction with a display device, illustrating software programs for patient care according to an embodiment of the present invention;

FIG. 7 shows sagittal, coronal and transverse anatomical planes of a human body;

FIGS. 8 and 9 show front views of a pelvis and an upper limb, respectively, of a human body;

FIGS. 10A and 10B show side views of Subject A with a pelvic sensor and a femoral sensor according to an embodiment of the present invention, and illustrating pelvic and femoral positions in standing and sitting with respect to the sagittal plane of Subject A's body;

FIGS. 11A and 11B show side views of Subject A with a pelvic sensor and a tibial sensor according to an embodiment of the present invention, and illustrating pelvic and tibial positions in standing and sitting with respect to the sagittal plane of Subject A's body;

FIGS. 12A and 12B show side views of Subject B with a pelvic sensor and a femoral sensor (FIG. 12A) or tibial sensor (FIG. 12B) according to an embodiment of the present invention, and illustrating pelvic, femoral and tibial positions in sitting with respect to the sagittal plane of Subject B's body;

FIGS. 13A-D show rear views of Subject C with a pelvic sensor and a femoral sensor according to an embodiment of the present invention, and illustrating pelvic and femoral positions in standing on two legs (FIG. 13A), standing on one leg (FIGS. 13B-D) and illustrating femoral abduction (FIG. 13C) and femoral adduction (FIG. 13D) with respect to the coronal or lateral plane of Subject C's body;

FIGS. 14A and 14B show cross-sectional views of a pelvis of Subject D with a pelvic sensor according to an embodiment of the present invention, and illustrating pelvic positions with respect to the transverse or axial plane when Subject D is standing on two legs;

FIG. 15 shows a perspective view of a pelvis of Subject E with a pelvic sensor and a femoral sensor according to an embodiment of the present invention, and illustrating an axis of rotation when Subject E is standing on one leg.

FIGS. 16A-B show Subject F with a femoral sensor according to an embodiment of the present invention, and illustrating femoral positions of external rotation (FIG. 16A) and internal rotation (FIG. 16B) with respect to the transverse or axial plane of Subject E's body;

FIGS. 17A-B show Subject G with an EMG sensor for measuring muscle activity of Subject G's gluteus medius in standing on two legs (FIG. 17A) and standing on one leg (FIG. 17B) according to an embodiment of the present invention;

FIG. 18 shows a side view of a pelvis of a human body;

FIGS. 19A-C show side views of a pelvis of a human body illustrating posterior (FIG. 19A), neutral (FIG. 19B) and anterior (FIG. 19C) pelvic positions in the sagittal plane of the human's body;

FIGS. 20A-C and 21 show a prior art classification method for classifying three pelvic positions in standing with respect to the sagittal plane of a human's body⁴;

FIGS. 22 and 23 show simplified classification charts for torso and limb positions of a vertebral mammal according to an embodiment of the present invention;

FIG. 24 shows a flow chart for a simplified classification algorithm for pelvic and femoral positions of a vertebral mammal according to an embodiment of the present invention;

FIGS. 25 and 26 show flow charts for a classification algorithm for pelvic and femoral positions of a vertebral mammal according to an embodiment of the present invention, with respect to the sagittal, coronal and transverse planes of the mammal's body.

### DETAILED DESCRIPTION

Embodiments of the invention are discussed herein by reference to the drawings which are not to scale and are intended merely to assist with explanation of the invention. The present invention relates to an apparatus and method for measuring and classifying positions of a torso and a limb of a body of a vertebral mammal. The vertebral mammal may include a human or an animal. The torso may include a pelvis or a spine of the mammal. When the torso includes the pelvis, the limb may include a lower limb of the mammal. The lower limb may include a femur or a tibia of the mammal, and preferably includes the femur. When the torso includes the spine, the limb may include an upper limb of the mammal. The upper limb may include a radius, an ulna or a humerus of the mammal, and preferably includes the humerus.

The inventive apparatus and method is useful for measuring and classifying positions of the torso and limb of a body of a human or animal subject, which may include patients with musculoskeletal conditions. For example, the classified positions of the torso and limb may provide an indication of stability of a hip or a shoulder joint, which may be useful for planning/evaluating hip or shoulder replacement surgery. Additionally/alternatively, the classified positions of the torso and limb may provide an indication of range of motion of the respective body parts, which may be useful for evaluating a risk of injury, such as in the workplace, to the spine or upper limb.

The inventive apparatus and method may provide reporting and feedback to subjects or patients across a continuum of care, which includes the patient journey from first symptoms, to progression through disease state, to early detection, assessment, diagnosis, treatment/s, exercise prescription, rehabilitation and ongoing monitoring, as shown by the software programs illustrated in FIG. 6.

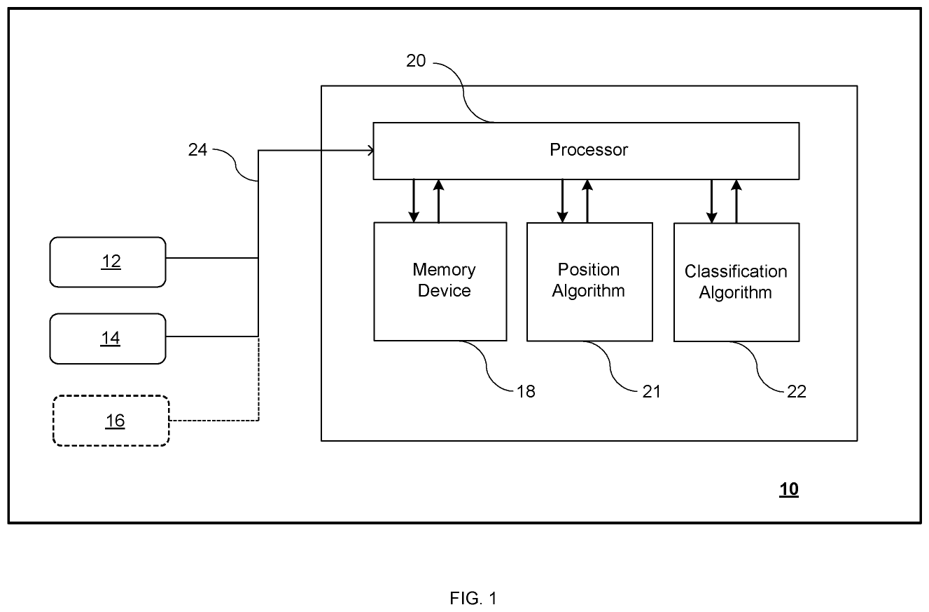

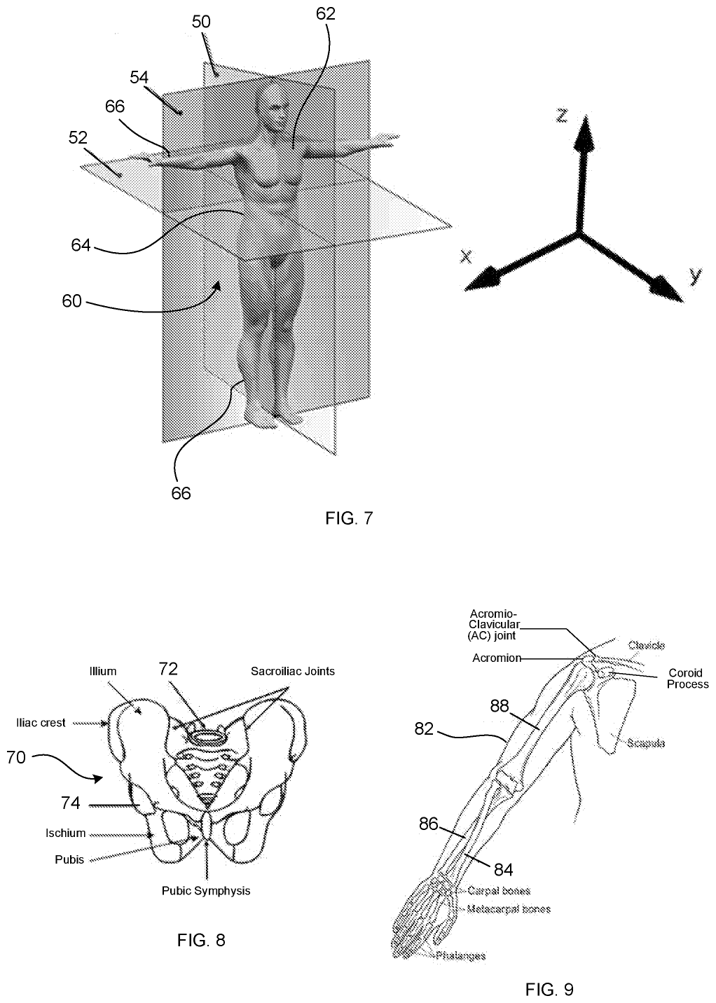

FIG. 1 shows an apparatus 10 for providing classification of position of a torso 64 and a limb 66 of a body 62 of a vertebral mammal 60 (see FIG. 7) according to a preferred embodiment of the present invention. The apparatus 10 includes a first sensor 12 for measuring position of the torso 64 relative to a first frame of reference and for providing first data indicative of the torso position. The apparatus 10 also includes a second sensor 14 for measuring position of the limb 66 relative to a second frame of reference and for providing second data indicative of the limb position. A memory device 18 of the apparatus 10 is adapted for storing the first and second data at least temporarily. A processor 20 of the apparatus 10 is adapted for processing the first and second data to derive angular positions of the torso 64 and the limb 66 in at least one anatomical plane (see FIG. 7, 50-54) of the mammal's body 62, and to provide the classification based at least on the derived torso and limb positions.

The positions of the torso 64 and limb 66 are measured by the first and second sensors 12 and 14 relative to first and second frames of reference, respectively. Preferably, the first and second sensors 12 and 14 are inertial sensors and the first and second frames of reference are inertial frames of reference, where the position measurements are made with respect to gravity. An inertial frame of reference denotes a frame of reference in which Newton's laws of motion apply. When no force is being exerted on an object then the object will move inertially. A frame of reference that moves with such an object is an inertial frame of reference. An inertial sensor denotes a sensor that responds to inertial forces such as forces that relate to acceleration of a system or that give rise to a change in velocity.

The first and second sensors 12 and 14 may be adapted to measure acceleration along one or more orthogonal axes. The first and second sensors 12 and 14 may include at least one acceleration sensor, such as an accelerometer, for measuring acceleration along one or more orthogonal axes. The first and second sensors 12 and 14 may be adapted to measure rotation around one or more orthogonal axes. The first and second sensors 12 and 14 may include at least one rotation sensor, such as a gyroscope, for measuring rotation around one or more orthogonal axes. Acceleration and/or rotation may be measured along or around one, two or three orthogonal axes, and the measurements may occur simultaneously.

The first and second sensors 12 and 14 may include at least one of an accelerometer, a gyroscope and a magnetometer. Preferably, the first and second sensors 12 and 14 include at least one accelerometer. The accelerometer may measure linear acceleration of the body or body part with which it is associated. The accelerometer may measure acceleration simultaneously along one, two or three orthogonal axes. The first and second sensors 12 and 14 may also include at least one gyroscope. The gyroscope may measure angular velocity or speed of the body or body part with which it is associated. The gyroscope may measure angular velocity or speed simultaneously along one, two or three orthogonal axes.

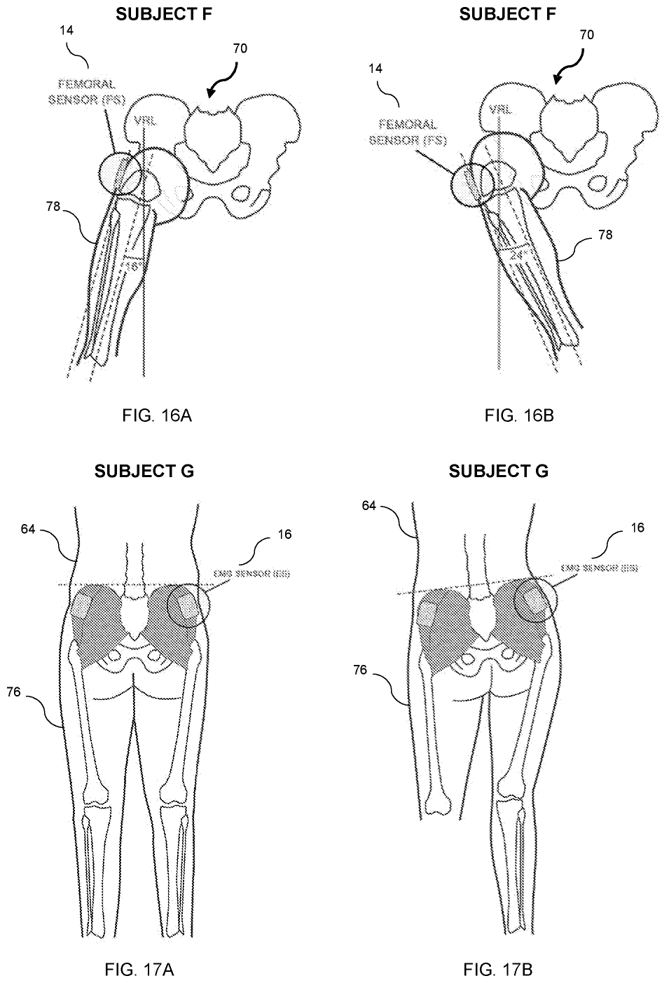

The apparatus 10 may also include a third sensor 16 for measuring muscle activity of the torso 64 and/or limb 66 of the mammal 60, and provide third data indicative of the muscle activity. The third sensor 16 may measure surface electromyography (EMG) to establish electrical activity within a muscle of the mammal 60. The measure of EMG may be correlated with muscle activity and used to calculate muscle fatigue. The processor 20 may be adapted to execute a muscle fatigue algorithm for calculating fatigue level. The muscle measuring sensor 16 preferably measures muscle activity in a lumbar back region of the mammal 60. For example, the third sensor 16 may be positioned on the lumbar spine 68 or pelvis 70. In some embodiments, a third sensor 16 may be positioned on the pelvis 70 at two locations to measure muscle activity, such as of the gluteus medius muscle, as shown in FIGS. 17A-B. Additionally, muscle activity may be measured in the biceps so as to assist in resolving ambiguous muscle activity readings from the back or pelvic muscles. This is particularly useful during movements of the mammal 60 from a fully bent over position to an upright position. Additionally, muscle activity may be measured at the Vastus Medialis Oblique (VMO) to provide inputs on knee stability and/or muscle fatigue during functional movements.

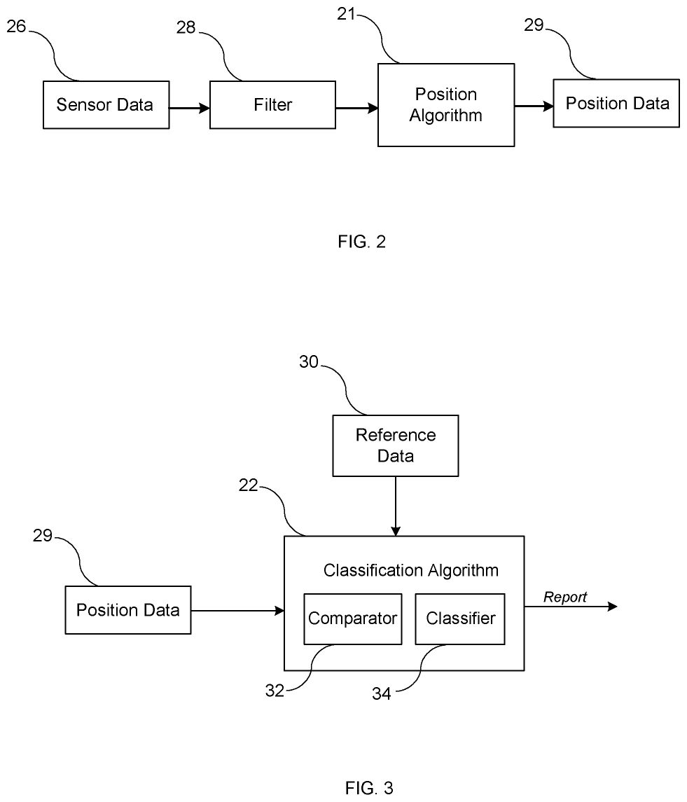

The sensors 12, 14 and 16 may be electronic sensors including wireless capabilities, a processor 20 and data storage capabilities 18. Alternatively, the sensors 12, 14 and 16 may store data internally or transfer data to an external source, such as through a wired or wireless link 24 (e.g., a storage device such as a USB) to a computing device or processor 20 (e.g., mobile device, tablet, personal computer or watch) as shown in FIG. 1. The sensors 12, 14 and 16 and/or other device such as processor 20 may process the data 26 from the sensors 12, 14 and/or 16 as shown in FIG. 2. The sensor data 26 (such as data from the first and second sensors 12 and 14 and/or sensor 16) may be filtered through filter 28 by the processor 20 to provide position data 29. The filtered data in the form of position data 29 may be provided as an input to a classification algorithm 22 as shown in FIG. 3.

The processor 20 is adapted to process the first and second data from the first and second sensors 12 and 14 to derive angular positions of the torso 64 and limb 66 in at least one anatomical plane of the mammal's body. The at least one anatomical plane may include the sagittal plane 50, transverse plane 52 and coronal plane 54 as shown in FIG. 7. The position data 29 provided to the classification algorithm 22 shown in FIG. 3 may include the derived angular positions of the torso 64 and limb 66.

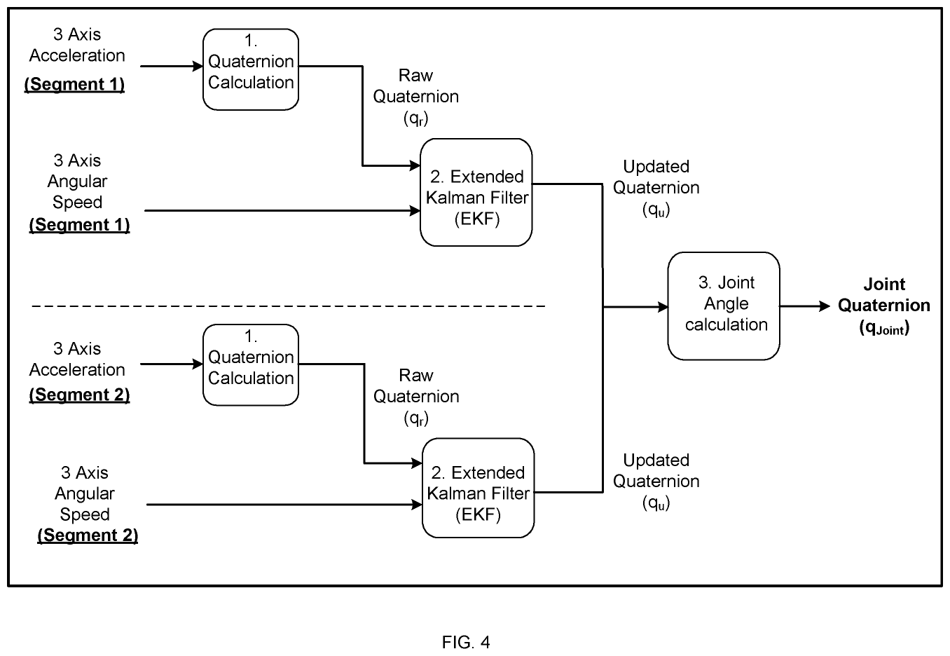

Referring again to FIGS. 1 and 2, the processor 20 may be further adapted to execute a position algorithm 21 for deriving an angular position of the limb 66 relative to the torso 64 in at least one anatomical plane of the mammal's body 62. The position algorithm 21 may transform the second data from the second frame of reference relative to the first frame of reference to derive the angular position of the limb 66 relative to the torso 64. The process for deriving angular positions of the torso 64 and limb 66 will be described below in more detail, particularly with reference to the position algorithm 21 shown in FIG. 4.

The processor 20 may be further adapted to execute the classification algorithm 22 as shown in FIGS. 1 and 3. The classification algorithm 22 may provide the classification based at least on the torso and limb positions derived by the processor 20. In addition, the processor 20 may be further adapted to receive reference data 30, which is provided as an input to the classification algorithm 22 as shown in FIG. 3. The reference data 30 can include one or more threshold values or a range of values for the torso and limb positions in at least one anatomical plane of the mammal 60 based on a normative population of vertebral mammals (see for example Table 2). The reference data 30 may be received by the processor 20 from a memory device 18 as shown in FIG. 1 or alternatively, from a remote database or server. The reference data 30 may be provided or filtered to be specific to humans or animals, and specific to demographics of the mammal 60, such as gender, height, weight, BMI and age, to name a few.

As shown in FIG. 3, the classification algorithm 22 may include a comparator 32 adapted to compare the derived torso and limb positions from the processor 20 to the reference data 30. The classification algorithm 22 also may include a classifier 34 adapted to classify the derived torso and limb positions based on the comparison by the comparator 32. For example, the classifier 34 may classify the derived torso and limb positions when they are more than or less than the threshold values or fall within the range of values of the torso and limb positions from the reference data 30. The classification algorithm 22 may also be further adapted to assign a class signature based on the classification of the derived torso and limb positions. The class signature may include a class number and/or the classified torso and limb positions, and further, may be indicative of stability of a hip and/or a shoulder joint of the mammal 60. The classification algorithm 22 will be described below in more detail.

The classification may be provided in static and/or dynamic states of the torso 64 and limb 66 of the mammal 60. For example, static states may include standing, sitting and lying down, whereas dynamic states may include movements such as sit to stand, stair ascending/descending, pivot turns, jogging, walking, running, climbing or other activities.

The memory device 18 may receive sensor data 26 from the sensors 12, 14 and/or 16, and may store the reference data 30. The memory device 18 may also receive position data 29 for storage. Each sensor 12, 14 and 16 may include an analog to digital (A to D) converter. Alternatively, the sensors 12, 14 and 16 may output analog data. The memory device 18 may include one or more A to D converters to convert the analog data to a digital domain prior to storing the sensor data 26 and/or position data 29. The memory device 18 may store the sensor data 26 and/or position data 29 in digital format, such as for analysis and/or reporting at a later time. The memory device 18 may include a card, stick or the like for storing digital data. Further, the memory device 18 may be removable from the apparatus 10 to facilitate downloading of the data to a remote processing device, such as a personal computer (PC) or mobile communication device, or uploading of the reference data 30.

Preferably, the processor 20 is a digital processor for processing the sensor data 26 (i.e. data from the sensors 12, 14 and/or 16) and for executing the position algorithm 21 and classification algorithm 22. The processing by processor 20 may be performed in real-time to provide feedback on the classification to a user. The user may be the subject, namely mammal 60, or a medical practitioner or surgeon. The processor 20 may also be adapted to generate an automated report on the classification, which includes the classified torso and limb positions and/or the class signature.

The apparatus 10 may include a user interface, such as display screen 36 (see FIG. 6) and one or more controls such as buttons or the like to allow the user to interact with the memory device 18. For example, the user interface may be provided by a mobile communication device, such as a laptop, smart phone or tablet.

The processor 20 may also be adapted to execute an algorithm for performing calculations based on risk assessment principles, which may include evaluation of risk components associated with individual data provided by each of the sensors 12, 14 and/or 16. Risk components may include profile data associated with the mammal being monitored, such as personal data and family history, which may have a bearing on risk of spine or limb injury and/or deterioration. The algorithm may evaluate risk of one or both of pelvic and lower limb injury and/or deterioration by using at least the third data from the muscle sensor 16. The evaluation may be cumulative to provide bio-feedback and used as a warning of impending risk and/or retraining system for rehabilitation of an existing injury. Risk components may be combined in accordance with risk principles to provide a cumulative evaluation of risk of spine or limb injury and/or deterioration. The risk components may be combined in a linear or non-linear fashion.

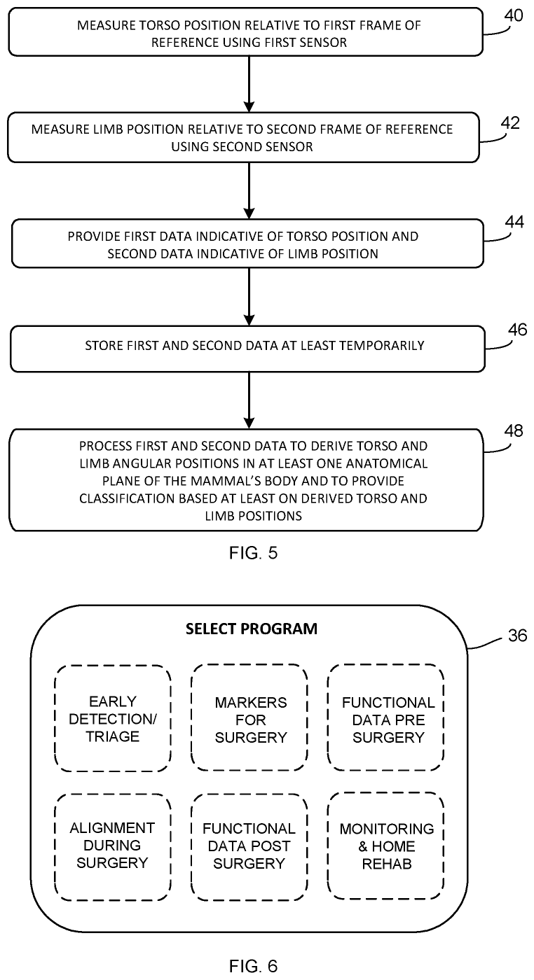

FIG. 5 shows a flow chart of a method for providing classification of position of a torso 64 and a limb 66 of a body 62 of a vertebral mammal 60 according to a preferred embodiment of the invention. The method includes at step 40 measuring position of the torso 64 relative to a first frame of reference using a first sensor 12 and at step 42 measuring position of the limb 66 relative to a second frame of reference using a second sensor 14. At step 44, the method includes providing first data indicative of the torso position and second data indicative of the limb position. The first and second data is stored at least temporarily at step 46. The method also includes at step 48 processing the first and second data to derive angular positions of the torso 64 and the limb 66 in at least one anatomical plane (See FIG. 7, 50-54) of the mammal's body 62, and to provide the classification based at least on the derived torso and limb positions.

The inventive method of FIG. 5 may include any of the steps performed by the processor 20 as described herein with reference to the apparatus 10. The first and second data may be stored at least temporarily at step 46 in a memory device 18 as shown and described with reference to FIGS. 1 to 4. The processing step 48 may include performing a position algorithm 21 for deriving an angular position of the limb 66 relative to the torso 64 in at least one anatomical plane of the mammal's body 62. The performing of the position algorithm 21 may include transforming the second data from the second frame of reference relative to the first frame of reference to derive the angular position of the limb 66 relative to the torso 64. The processing step 48 may further include performing a classification algorithm 22 for providing the classification based at least on the derived torso and limb positions. The position algorithm 21 and the classification algorithm 22 may be performed as described herein. The processing step 48 may include processing the first and second data using a processor 20 as shown and described herein with reference to FIGS. 1 to 4.

In some embodiments, the method includes receiving reference data 30 for use by the comparator 32 of the classification algorithm 22. Performing the classification algorithm 22 may further include the step of assigning a class signature based on the classification of the derived torso and limb positions as described herein.

The method may further include the step of generating an automated report on the classification, where the report includes one or both of the classified torso and limb positions and the class signature. The method may perform the classification in static and/or dynamic states of the torso 64 and limb 66. The processing step 48 may be performed in real-time to provide feedback on the classification to a user.

In some embodiments, each measuring step 40 and 42 includes converting analog data to a digital domain using at least one analog to digital (A to D) converter of the first and second sensor 12 and 14. Preferably, the A to D conversion takes place prior to storing the first and second data. The method may also include a step of measuring muscle activity of the torso 64 and/or limb 66 of the mammal 60 from a third sensor 16, and providing third data indicative of the muscle activity.

FIG. 6 shows use of the inventive apparatus 10 and method in conjunction with software on a mobile communication device, having a display device 36, and demonstrating use across a continuum of care for patients with musculoskeletal conditions. The findings from these assessments may be able to identify patients requiring surgery, monitor movement and function over time and guide and report on rehabilitation (including home based) via an app based exercise library with inbuilt reminders.

### Sensor Placement Protocol

Use of the apparatus 10 requires locating of landmarks on the torso 64, and more specifically the spine 68 and pelvis 70, and on the limb 66, such as the lower limb 76 or upper limb 82, to ensure reliable readings from the first and second sensors 12 and 14.

The landmarks may be located on the lumbar spine 68 and pelvis 70 using the following procedure:
1. Locate the Posterior Superior Iliac Spine (PSIS) by palpating the superior aspect of the iliac spine of the subject 60.
2. When located, mark the PSIS by drawing small circles over them.
3. Use a ruler to draw a horizontal line through the centres of the PSIS circles.
4. Place a pelvic sensor 12 on the subject's body 62 directly underneath the line, or using a Low Back fitment template, place a spinal sensor 12 on the subject's body 62 so it sits at T12/L1 level of the spine 68. The sensor 12 should be placed in landscape orientation.

The landmarks may be located on the femur 78 using the following procedure:
1. Ask the subject 60 to sit on the edge of a chair with hips and knees in 90 degree flexion and mark the top and inferior border of the patella.
2. Measure and mark the mid-point with a “cross-hair”.
3. With a tape measure (and the hip and knee still flexed 90 degrees), draw a line towards the greater trochanter from that point and measure 10 cm from the cross-hair to mark the centre of where the sensor 14 should be replaced.
4. Place the sensor 14 in landscape orientation so an upper edge of the sensor 14 is aligned with that line and its end is placed on the 10 cm mark.

The landmarks may be located on the tibia 80 using the following procedure:
1. Using a Tibia Template, place the zero line of the ruler of the inferior border of the medial malleolus and mark the shin bone at the appropriate height demarcation.
2. Place the sensor 14 on the tibia 80 in portrait orientation so it sits in the middle of the line drawn on the shin bone.

The apparatus 10 of the present invention should be accurately fixed to the torso 64 and limb 66 to minimise reading errors. Care should be taken in fixing transducer pads which may store the first and second sensors 12 and 14 and EMG electrode assembly 16.

Fixation preferably should adhere to the following precautions:
1. Determine if the subject 60 has skin allergies prior to application of sensors 12 and 14 and EMG sensor 16. If so, a protective film may be applied to skin prior to sensor application. If the subject 60 has severe allergies, do not use.
2. Care must be taken when applying sensors 12, 14, 16 to fragile skin of the subject 60.
3. Only use the apparatus 10 for purposes for which it is intended.
4. In order to reduce the risk of skin irritation, it is advised not to wear the sensors 12, 14 and 16 for more than 24 hours in any 72 hour period.

### Deriving Torso and Limb Positions

Angular position or deviation data of the torso 64 and limb 66 may be derived by the processor 20 from the first and second sensors 12 and 14 by a process of integration as is well known in the art, such as by using accelerometer, gyroscope and/or magnetometer data. Alternatively/additionally data may be derived by the processor 20 to provide angular position or deviation relative to a reference such as a direction defined by gravity, such as by using accelerometer, gyroscope and/or magnetometer data. The processor 20 may be adapted to derive angular position or deviation from acceleration data by integrating linear acceleration data to provide linear position data and calculating a forward tilt angle and a side tilt angle. The first and second sensors 12 and 14 may also include a gyroscope for deriving rotational position of the respective body part. The rotational position may be derived by double integration of angular velocity or speed data to provide angular position.

In particular, the first and second sensors 12 and 14 may include at least one accelerometer for measuring acceleration of the respective body part. Each accelerometer may detect a change in acceleration of a small mass mounted within a micro chip on a PCB board. As the PCB board, and the accelerometer move from one position to another, the mass experiences an acceleration at the start of the movement as well as a deceleration as the movement ceases. The accelerometer may convert movement of the mass into a voltage signal (typically in mV) that represents data in its most raw form.

Span and offset adjustments may convert the voltage signal to a G force value, by way of calibration constants. A first calibration constant (p) is known as a ‘multiplier’ or ‘gain’ constant and may be derived by means of simultaneous equations wherein signal values equate to G force values. A second calibration constant (o) is known as an offset constant. Once calculated, the calibration constants (p) and (o) may be programmed into software and may become a permanent fixture of the programming. There may be two calibration constants for each channel and three channels per sensor 12 and 14.

Angular displacement of the accelerometer may be calculated by multiplying the raw signal value by the gain constant (p) and adding the offset constant (o). The resulting value may represent the G force acting on one axis. For a resultant G force in three dimensions, three axes trigonometry may be used, wherein x is the horizontal axis, y is the vertical axis and z is the ‘through page’ axis. Using 3D Pythagoras and an inverse tangent formula, two angles may be derived to give a position for the accelerometer. One accelerometer in isolation may only give a direction of movement, but when there are two accelerometers, the difference between angles of the two accelerometers may represent a change in position (in degrees) of one accelerometer compared to the other accelerometer. This may allow the apparatus 10 to calculate angular position or deviation of the torso 64 and limb 66, and more specifically, the spine 68, pelvis 70, lower limb 76 including the femur 78 and/or tibia 80, and the upper limb 82 including the radius 84, ulna 86 and/or humerus 88 (as shown in FIGS. 7-9), at any moment in time, within a three-dimensional axis.

The following expressions may be used to derive angular changes from accelerometers.

ep+o=1g

fp+o=−1g

where:
e=millivolts for 1 g;
f=millivolts for −1 g;
p=gain (multiplier); and
o=offset
   solving p and o:

$$ep + o - fp - o = 2 g \\ ( e - f ) p = 2 g$$

$$p = \frac{2 g}{e - f}$$

$$ep + o = 1 g$$

$$o = 1 g - ep$$

$$or$$

$$fp + o = - 1 g$$

$$o = - 1 g - fp$$

Note: values for p and o should be calculated for each axis.

x_{mV}pₓ+oₓ=x_{g}

y_{mV}p_{y}+o_{y}=y_{g}

z_{mV}p_{z}+oₓ=z_{g}

The above 3 equations show for the 3 axes the span and offset adjustment which converts millivolts to g.

The magnitude and tilt (forward/side) for the resultant vectors may be calculated as follows.

### Magnitude:

r_{g}=\sqrt{x_{g}²+y_{g}²+z_{g}²}

The magnitude represents the vector sum in three dimensions of the resultant G force.

### Forward Tilt:

$$θ = {tan}^{- 1} ( \frac{Zg}{\sqrt{{x}_{g}^{2} + {y}_{g}^{2}}} )$$

### Side Tilt:

$$β = {tan}^{- 1} ( \frac{Xg}{\sqrt{{z}_{g}^{2} + {y}_{g}^{2}}} )$$

The forward and side tilt angles θ, β give the rotational position of the accelerometer relative to the z and x axes respectively. Accordingly, the angular position of the torso 64 and limb 66 in the sagittal plane 50 and coronal plane 54 may be derived using the forward and side tilt angles from the accelerometer data.

In some embodiments, the first and second sensors 12 and 14 may include at least one rotational sensor, such as a gyroscope, for measuring rotational position of the torso 64 and limb 66 in the transverse plane 52. In some embodiments, a measure of rotation may also be derived from one or more accelerometers, muscle activity and/or one or more gyroscopes.

Alternatively, the torso and limb angular position data may be acquired by means of at least one magnetometer sensor. Each magnetometer may measure strength and/or direction of the earth's magnetic field by a change or changes in resistance of a thin film deposited on a silicon wafer (anisotropic magnetoresistive magnetometers) or by a change or changes in a coil on a ferromagnetic core (magnetoinductive magnetometers). The coil may include a single winding and may form an inductance element in a L/R relaxation oscillator. A magnetometer may measure strength and/or direction of the earth's magnetic field in one, two or three planes. Earth's North may be used as a reference to compute orientation of a body with assistance of three axis trigonometry.

Preferably, an angular position of the limb 66 is derived relative to the position of the torso 64 in at least one anatomical plane of the mammal's body 62. In order to determine the limb angular position, the second data from the second frame of reference may be transformed to the first frame of reference for the torso 64. This process of data transformation is well known to a person skilled in the art.

In order to determine the angular position of the limb 66, a joint angle calculation may be performed, using two segments of data measured from second sensors 14 positioned at two locations on the limb 66. For example, in measuring angular position of the lower limb 76, the second sensors 14 may be positioned on the femur 78 and tibia 80. The second sensors 14 may include at least one acceleration sensor, such as an accelerometer, for providing acceleration data along at least three orthogonal axes, and at least one rotation sensor, such as a gyroscope, for providing angular speed or angular velocity data along at least three orthogonal axes.

Referring now to FIG. 4, the joint angle calculation involves firstly calculating a Raw Quatemion (qᵣ) for each data segment using the three-axis acceleration data. Secondly, an Extended Kalman Filter (EKF) is applied to the calculated Raw Quaternions (qᵣ) using the three-axis angular speed or velocity data to calculate the Updated Quaternion (qᵤ). The EKF combines positional estimates from the acceleration and velocity data to reduce integration errors that may occur and to resolve drift errors in using the gyroscope data. Thirdly, the Updated Quaternions (qᵤ) from the two segments of data are used in a joint angle calculation to calculate the Joint Quatemion (qⱼₒᵢₙₜ).

The following expressions may be used to derive the Joint Quatemion (qⱼₒᵢₙₜ).

### Acceleration to Quaternion Equations:

$$\{ \begin{matrix}\begin{matrix}{q}_{0} = \sqrt{\frac{{a}_{z} + 1}{2}} \\ {q}_{1} = \frac{- {a}_{y}}{\sqrt{2 \cdot ( 1 + {a}_{x} )}} \\ {q}_{2} = \frac{{a}_{x}}{\sqrt{2 \cdot ( 1 + {a}_{z} )}} \\ {q}_{3} = 0\end{matrix} \} {a}_{z} > 0 \\ \begin{matrix}{q}_{0} = \frac{- {a}_{y}}{\sqrt{2 \cdot ( 1 - {a}_{z} )}} \\ {q}_{1} = \sqrt{\frac{1 - {a}_{z}}{2}} \\ {q}_{2} = 0 \\ {q}_{3} = \frac{{a}_{x}}{\sqrt{2 \cdot ( 1 - {a}_{z} )}}\end{matrix} \} {a}_{z} < 0\end{matrix}$$

### Acceleration to Dip Angle Equations:

$$\{ \begin{matrix}ϕ = {tan}^{- 1} ( \frac{{a}_{y}}{- {a}_{x}} ) \times 180 \text{/} π \\ θ = {tan}^{- 1} ( \frac{{a}_{z}}{\sqrt{{a}_{x}^{2} + {a}_{y}^{2}}} ) \times 180 \text{/} π \\ Ψ = 0\end{matrix}$$

where qᵣ=[q₀,q₁,q₂,q₃] is the Raw Quatemion (qᵣ), a=[aₓ,a_{y},a_{z}] are the raw accelerations

### Extended Kalman Filter (EKF):

$$z = \{ \begin{matrix}{e}^{- \frac{Δ t}{τ}} \cdot {x}_{1} \\ {e}^{- \frac{Δ t}{τ}} \cdot {x}_{2} \\ {e}^{- \frac{Δ t}{τ}} \cdot {x}_{2} \\ {x}_{4} - \frac{Δ t}{2} ( {x}_{1} {x}_{5} + {x}_{2} {x}_{6} + {x}_{3} {x}_{7} ) \\ {x}_{5} + \frac{Δ t}{2} ( {x}_{1} {x}_{4} - {x}_{2} {x}_{7} + {x}_{3} {x}_{6} ) \\ {x}_{6} + \frac{Δ t}{2} ( {x}_{1} {x}_{7} + {x}_{2} {x}_{4} - {x}_{3} {x}_{5} ) \\ {x}_{7} + \frac{Δ t}{2} ( - {x}_{1} {x}_{6} + {x}_{2} {x}_{5} + {x}_{3} {x}_{4} )\end{matrix}$$

where qᵤ=[x4,x5,x6,x7] is the Updated Quaternion (qᵤ) and g=[x1,x2,x3] are gyroscope readings.

qⱼₒᵢₙₜ=(q̅_̅{̅c̅a̅l̅1̅}̅·̅q̅_̅{̅s̅e̅g̅1̅}̅)·(q̅_̅{̅c̅a̅l̅2̅}̅·q_{seg2}) Joint angle calculation:

where q_{Seg1} and q_{Seg2} correspond to the two different segments and q_{cal1} and q_{cal2} correspond to the calibration quaternions to the anatomical frame of reference.

The processor 20 may be adapted to process the first and second data to derive angular positions of the torso 64 and the limb 66 in at least one anatomical plane of the mammal's body. FIG. 7 shows the three orthogonal anatomical planes of a human body 62, namely the sagittal or anterior-posterior (front-to-back) plane 50, the transverse or axial plane 52 and the coronal or lateral (side-to-side) 54. The processor 20 may process the first data to derive at least angular position or deviation of the torso 64 in the sagittal plane 50 or the coronal plane 54, and to derive rotation of the torso 64 in the transverse plane 52. Similarly, the processor 20 may process the second data to derive at least one of angular position or deviation of the limb 66 in the sagittal plane 50 or the coronal plane 54, or to derive rotation of the limb 66 in the transverse plane 52. Preferably, the derived angular positions of the limb 66 are relative to the torso 64.

### Examples of Pelvic, Femoral and Tibial Positions

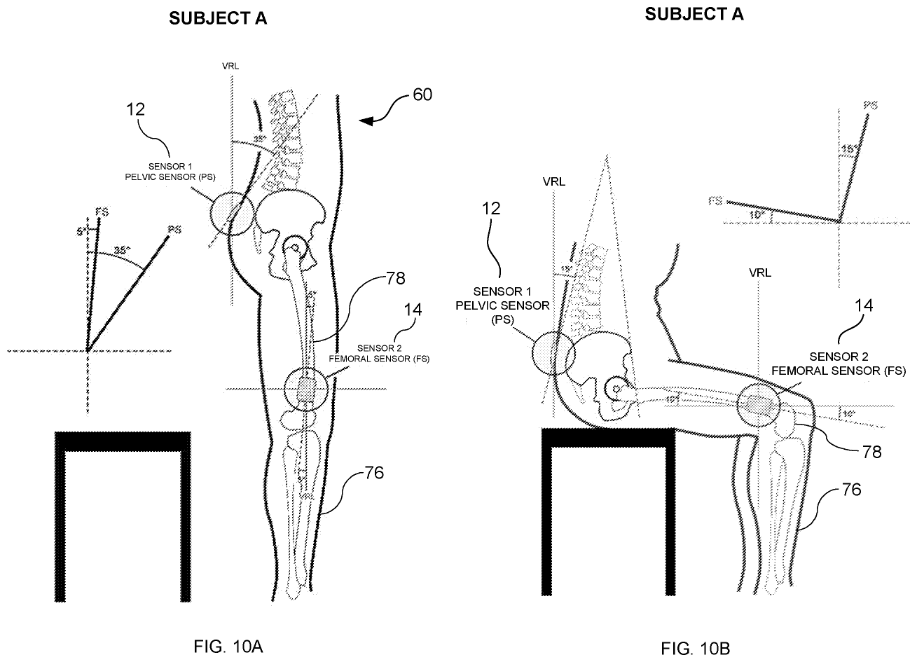

Examples of the derived angular pelvic, femoral and tibial positions in the sagittal, coronal and transverse planes 50-54 of a body 62 of a human 60, will now be described with reference to the embodiments shown in FIGS. 10 to 17. By way of background, FIGS. 8 and 9 show front views of a pelvis 70 and upper limb 82 with shoulder joint, respectively, of a human body 62. Referring to FIG. 8, the pelvis 70 includes three bones, namely the sacrum 72 and two hip bones on the left and right. The hip bones join the sacrum 72 via stiff fibrous joints called the sacroiliac joints (left and right side) and at the front (or anterior aspect) the two hip bones join at the pubic symphysis. The left and right sides of the pelvis 70 each include an acetabulum 74 for receiving a femoral head of the femur 78.

FIGS. 10A-B show side views of Subject A with a pelvic sensor 12 arranged on skin near the sacrum 72 and a femoral sensor 14 according to an embodiment of the invention. FIGS. 10A-B illustrate the pelvic and femoral positions in standing and sitting with respect to the sagittal plane 50 of Subject A's body 62. The pelvis 70 moves from 35° off vertical (in standing) to 15° off vertical in sitting for Subject A. The femur 78 moves from 5° off vertical (in standing) to 10° below the horizontal in sitting. The difference between the pelvic and femoral positions in standing is 30° whereas the difference between the pelvic and femoral positions in sitting is 85°. The positions of the pelvis 70 and femur 78 are derived from data measured by the first and second sensors 12 and 14. The angular position or deviation in the sagittal plane 50 is determined with respect to a Vertical Reference Line (VRL).

Table 1 below describes the variance in pelvic and femoral position when Subject A is standing and sitting. An example of a relationship between the pelvis 70 and femur 78 during an activity like stand to sit, would be looking at the ratio of pelvic movement compared to femoral movement (e.g., pelvis 12 moves 20° whereas the femur 16 moves 95°, a ratio of 20:95 or 1:4.75).

TABLE 1
Variance in pelvic and femoral positions for Subject or Patient A
Patient A (Sagittal plane only)

| | | | Difference between Pelvic |
|---|---|---|---|
| | Pelvis | Femur | position and femoral position |
| Standing | 35 | 5 | 30 |
| Sitting | 15 | 100 | 85 |
| Change in Pelvic or | 20 | 95 | |
| Femoral movement | | | |
| from stand to sit | | | |

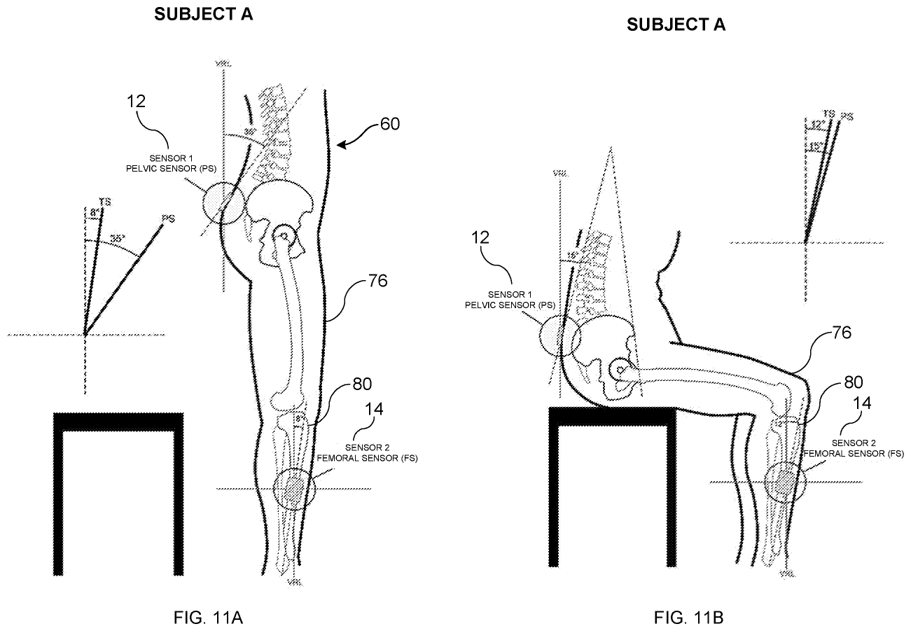

FIGS. 11A-B show side views of Subject A with a pelvic sensor 12 and a tibial sensor 14 according to another embodiment of the present invention. The figures illustrate the pelvic and tibial positions in standing and sitting with respect to the sagittal plane 50 of Subject A's body 62. As Subject A moves from a standing to seated position, the pelvis 70 displaces posteriorly from 35° to 15° and the tibia 80 flexes anteriorly from 8° to 12°.

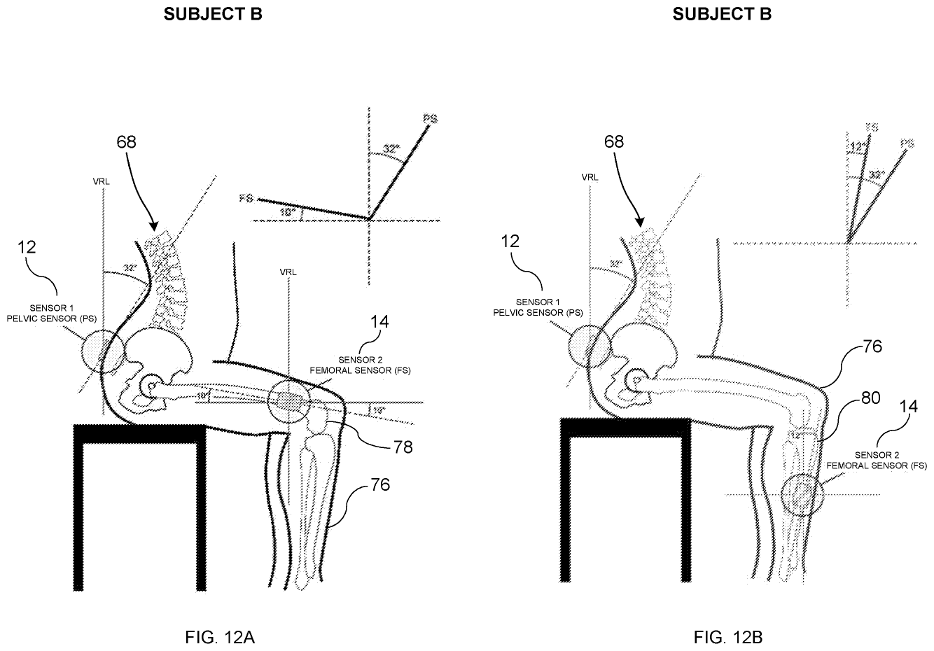

FIGS. 12A-B show Subject B with a pelvic sensor 12 and a femoral sensor 14 (FIG. 12A) or a tibial sensor 14 (FIG. 12B) according to an embodiment of the present invention. The figures illustrate pelvic, femoral and tibial positions in sitting with respect to the sagittal plane 50 of Subject B's body 62. The difference in positions is evident when Subject B is compared to Subject A (see FIGS. 10A and 10B). In particular, when Subject B is seated, the pelvis 70 is held in a significant amount of anterior tilt (32° compared to 15° in Subject A).

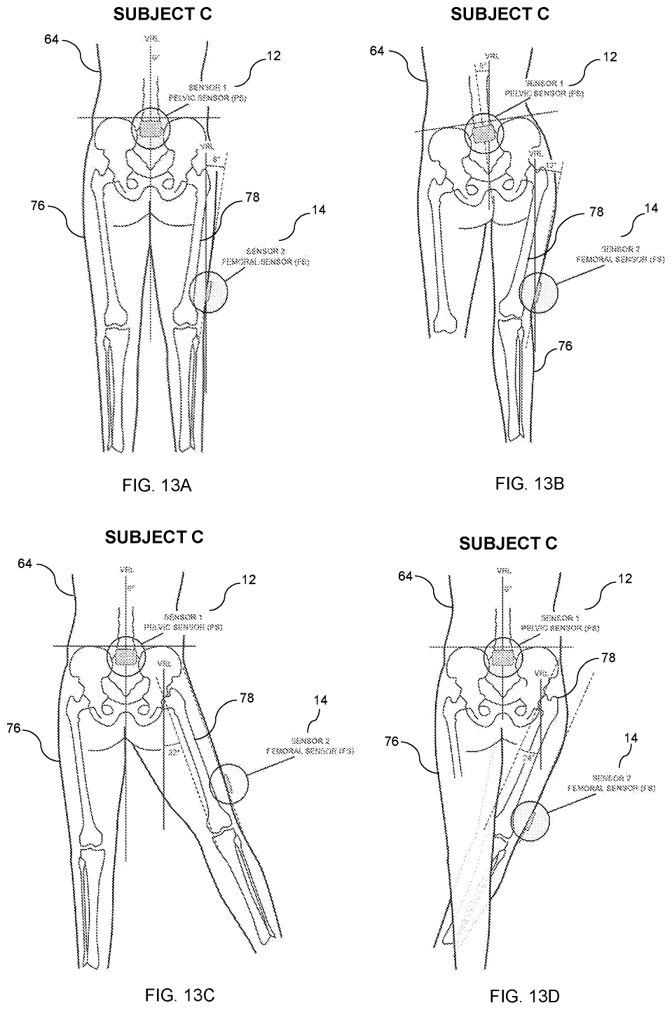

FIGS. 13A-D show rear views of Subject C with a pelvic sensor 12 and a femoral sensor 14 according to an embodiment of the present invention. The figures illustrate the pelvic and femoral positions in standing on two legs (FIG. 13A), standing on one leg (FIGS. 13B-D) and illustrating femoral abduction (FIG. 13C) and femoral adduction (FIG. 13D) with respect to the coronal or lateral plane 54 of Subject C's body 62. FIGS. 13A-B show that when Subject C moves from two legs onto one leg, the pelvis 70 displaces laterally to the left by 8°, and the femur 78 displaces in adduction from 8° to 12°. FIGS. 13A-C show that when Subject C moves the femur 78 in abduction, the femur 78 displaces laterally from 80 right to 22° left, a change of 30°. FIGS. 13A-D show that when Subject C moves the femur 78 in adduction, the femur 78 displaces laterally from 8° right to 24° right, a change of 16°.

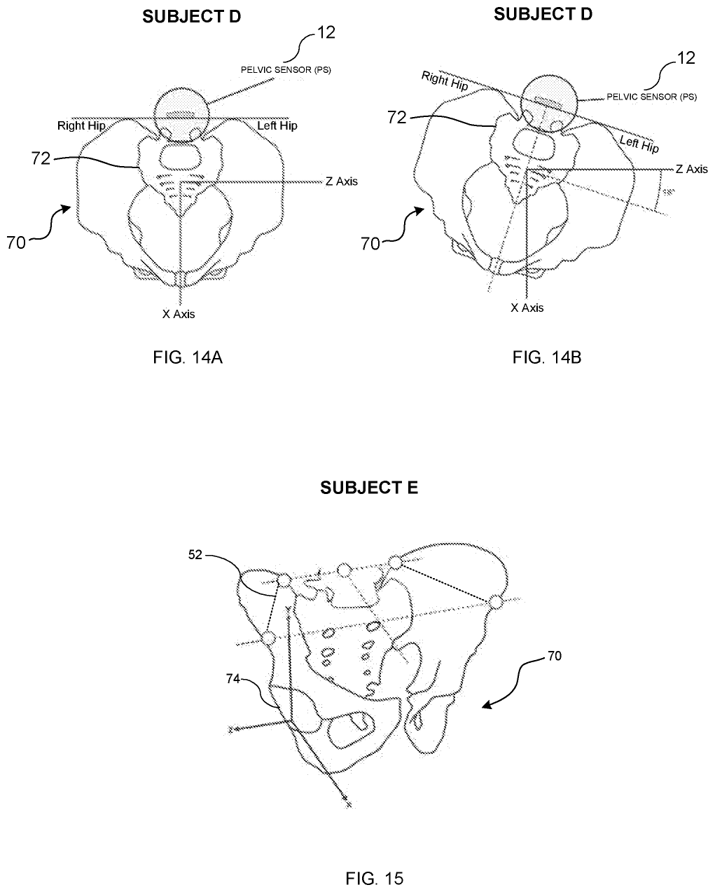

FIGS. 14A-B show cross-sectional views of a pelvis 70 of Subject D with a pelvic sensor 12 according to an embodiment of the present invention. The figures illustrate pelvic rotation in the transverse or axial plane 52 when Subject D is standing on two legs. The axis of rotation is through the central aspect of the pelvic ring/sacrum 72. When Subject D moves from the position of FIG. 14A to FIG. 14B, the pelvis 70 rotates right 18°.

FIG. 15 shows a perspective view of a pelvis 70 of Subject E with a pelvic sensor 12 (on sacrum) according to an embodiment of the present invention. The figure illustrates pelvic rotation in the transverse or axial plane 52 when Subject D is standing on one leg. The axis of rotation is more through the acetabulum 74, as shown by the axis near the right acetabulum 74 in FIG. 15.

FIGS. 16A-B show Subject F with a pelvis sensor 12 (on scarum 72) and a femoral sensor 14 according to an embodiment of the present invention. The figures illustrate femoral positions of external rotation (FIG. 16A) and internal rotation (FIG. 16B) with respect to the transverse or axial plane 52. In FIG. 16A, as Subject F rotates the tibia 80 outward, the femur 78 rotates internally by 16°. In FIG. 16B, as Subject F rotates the tibia 80 inward, the femur 78 rotates externally by 24°.

FIGS. 17A-B show Subject G with an EMG sensor 16 on the right and left gluteus medius muscles according to an embodiment of the present invention. The EMG sensor 16 measures muscle activity when Subject G is standing on two legs (FIG. 17A) and standing on one leg (FIG. 17B). EMG activity can be used as an indicator of strength, weakness and/or fatigue of muscle and/or spasm of muscle. The analysis of EMG may also review different frequency spectrums and sampling rates to identify patterns of muscle activity that may be clinically relevant. If there is significant weakness in the muscle of the right gluteus medius, the pelvis 70 will displace laterally left as shown in FIG. 17B, which is a clinical indication for muscle deficiency. For example, this weakness or reduced activity in the gluteus medius muscle is often evident in subjects with an arthritic hip joint.

Although the above examples of FIGS. 10 to 17 are limited to measuring and deriving pelvic, femoral and tibial angular positions in the sagittal, coronal and transverse planes 50-54, it would be appreciated by a person skilled in the art that similar positions could be measured and derived with respect to the spine 68 and upper limb 82 of the mammal 60. For example, the first sensor 12 could be positioned on the spine 68 and the second sensor 14 positioned on the upper limb 82, such as on the humerus 88, ulna 86 or radius 84. Thus, angular positions of the spine 68 and upper limb 82 may be measured and derived in the sagittal, coronal and transverse planes 50-54 of the mammal 60, using a similar process as described herein with reference to the pelvis 70, femur 78 and tibia 80.

### Classification Algorithm

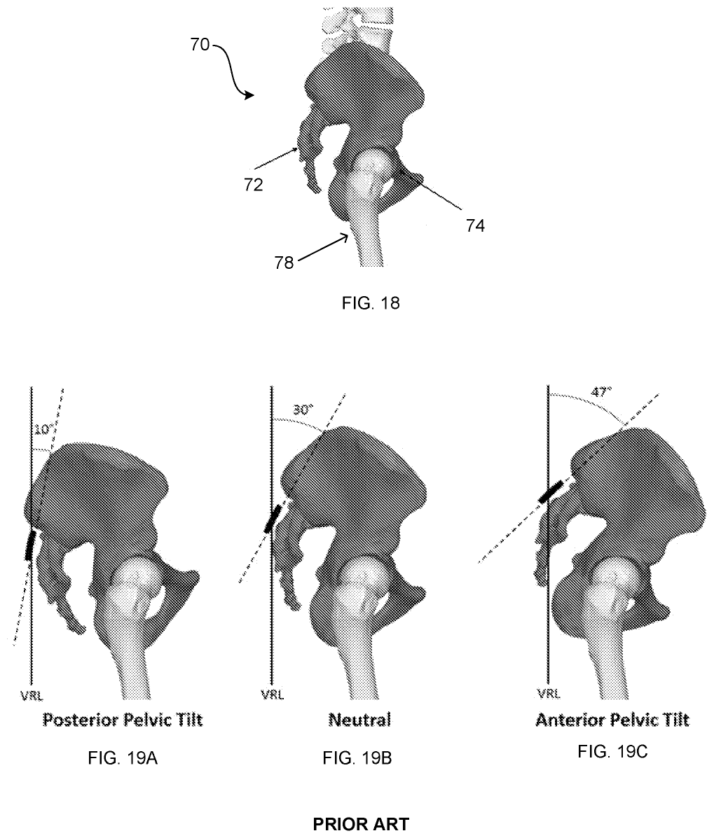

The classification algorithm 22 includes a comparator 32 adapted to compare the derived angular torso and limb positions in at least one anatomical plane to reference data 30 (see FIG. 3). The derived positions may include at least one of angular position or deviation in the sagittal or coronal planes 50 and 54, and rotation in the traverse plane 52, as shown for example in FIGS. 10 to 17. The comparator 32 compares these derived positions to reference data 30, which may include known angular ranges for classifying positions in the anatomical planes, as would be known and ascertainable by a person skilled in the art. An example of such reference data 30 will now be described with reference to FIGS. 18 to 21 and may include angular ranges for classifying pelvic positions in the sagittal plane 50 of the mammal's body 62.

FIG. 18 shows a side view of a pelvis 70 of a human body 62. The pelvis 70 is able to tilt backward or posterior tilt (FIG. 19A), forward or anterior tilt (FIG. 19B), or maintain a neutral position (FIG. 19C) in an extension-flexion or sagittal plane 50 of the human's body 62 (see FIG. 7). It is possible to assess the pelvic tilt by measuring an angle between a longitudinal axis of a marker positioned on the pelvis 12, such as near the sacrum 72 (dotted line) and a vertical reference line (VRL) (solid line). The size of the angle measured can be used to classify the pelvic tilt.

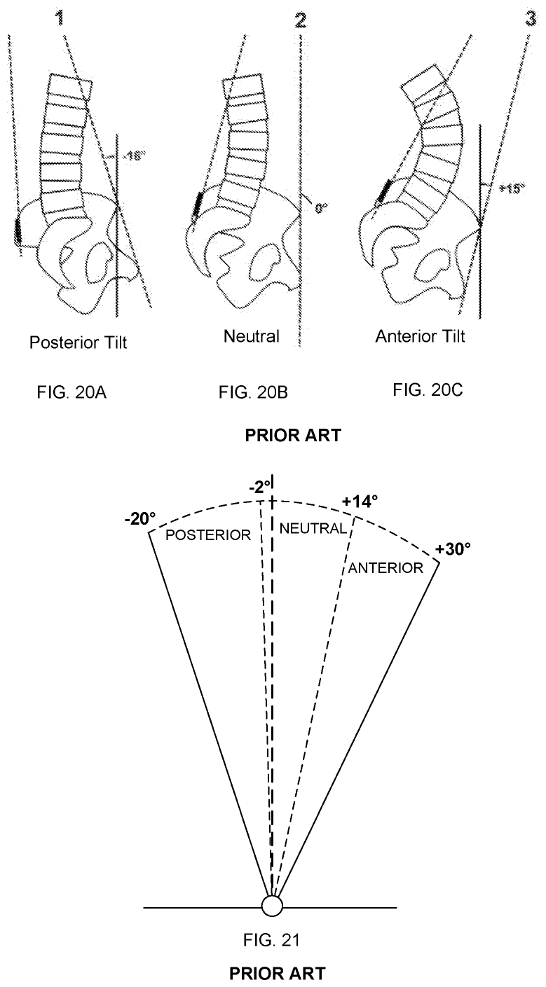

Referring to FIGS. 20A-C and 21, prior art angular ranges for classifying pelvic angular positions or tilt in standing is shown. The figures illustrate posterior tilt (FIG. 20A), neutral (FIG. 20B) and anterior tilt (FIG. 20C) in a standing position of a human's body 62. In this example, pelvic tilt is assessed by measuring an angle between a longitudinal axis based on anterior landmarks on the pelvis 70 and a vertical axis. Table 2 below illustrates an example of reference data 30 in the form of angular ranges for classifying a movement zone of the pelvis 70 as posterior, neutral or anterior, in the sagittal plane 50 of a human's body 62, by comparing the measured angle to normative ranges of angles representative of the movement zones.

TABLE 2
One example of prior art angular ranges for classifying
pelvic angular positions in the sagittal plane

| MOVEMENT ZONE | ZONE RANGE |
|---|---|
| Posterior | −20° to −2° |
| Neutral | −2° to +14° |
| Anterior | +14° to +30° |

Pelvic tilt is traditionally a difficult movement to detect and accurately measure due to the required angular measurements of the pelvis 70, as described with reference to the known methods of FIGS. 18 to 21. Accordingly, the apparatus 10 of the present invention and method as described herein may desirably provide the use of wearable sensors to measure position of the pelvis 70 with a high degree of accuracy and also to desirably provide for ease of use by the mammal or subject 60 and a medical practitioner, such as a surgeon.

Although the above example of FIGS. 18 to 21 for reference data 30 is limited to angular ranges for classifying pelvic position in the sagittal plane 50, it would be appreciated by a person skilled in the art that the reference data 30 could be threshold values, such as a threshold angle of angular position or deviation in the sagittal plane 50. It would also be appreciated by a person skilled in the art that reference data 30 could be ascertained from the literature or prior art with respect to the transverse and coronal planes 52 and 54. Furthermore, a person skilled in the art would be able to ascertain reference data 30, such as threshold values or ranges of values, with respect to the torso 64, lower limb 76 and upper limb 82, particularly for the spine 68, femur 78, tibia 80, radius 84, ulna 86 and humerus 88, and in each of the three anatomical planes 50-54 of the mammal's body 62.

The classification algorithm also includes a classifier 34 adapted to classify the derived torso and limb positions based on a result of the comparison. The result may include when the derived torso and limb positions are more than or less than the threshold value or within the range of values from the reference data 30. The classifier 34 may classify the derived torso and limb positions in the sagittal, coronal and/or transverse planes based on the classifications provided in Table 3 below.

TABLE 3
Torso and Limb Positions in 3-D Space

| Torso Positions in 3-D Space | | | Limb Positions in 3-D Space | | |
|---|---|---|---|---|---|
| Sagittal | Coronal | Transverse | Sagittal | Coronal | Transverse |
| Plane | Plane | Plane | Plane | Plane | Plane |
| (Tilt) | (Tilt) | (Rotation) | (Motion) | (Motion) | (Rotation) |
| Anterior | Left | Left | Flexion | Abduction | Internal |
| Anterior | Left | Neutral | Flexion | Abduction | Neutral |
| Anterior | Left | Right | Flexion | Abduction | External |
| Anterior | Neutral | Left | Flexion | Neutral | Internal |
| Anterior | Neutral | Neutral | Flexion | Neutral | Neutral |
| Anterior | Neutral | Right | Flexion | Neutral | External |
| Anterior | Right | Left | Flexion | Adduction | Internal |
| Anterior | Right | Neutral | Flexion | Adduction | Neutral |
| Anterior | Right | Right | Flexion | Adduction | External |
| Neutral | Left | Left | Neutral | Abduction | Internal |
| Neutral | Left | Neutral | Neutral | Abduction | Neutral |
| Neutral | Left | Right | Neutral | Abduction | External |
| Neutral | Neutral | Left | Neutral | Neutral | Internal |
| Neutral | Neutral | Neutral | Neutral | Neutral | Neutral |
| Neutral | Neutral | Right | Neutral | Neutral | External |
| Neutral | Right | Left | Neutral | Adduction | Internal |
| Neutral | Right | Neutral | Neutral | Adduction | Neutral |
| Neutral | Right | Right | Neutral | Adduction | External |
| Posterior | Left | Left | Extension | Abduction | Internal |
| Posterior | Left | Neutral | Extension | Abduction | Neutral |
| Posterior | Left | Right | Extension | Abduction | External |
| Posterior | Neutral | Left | Extension | Neutral | Internal |
| Posterior | Neutral | Neutral | Extension | Neutral | Neutral |
| Posterior | Neutral | Right | Extension | Neutral | External |
| Posterior | Right | Left | Extension | Adduction | Internal |
| Posterior | Right | Neutral | Extension | Adduction | Neutral |
| Posterior | Right | Right | Extension | Adduction | External |

Through the combined positions of the torso 64 and limb 66 as shown in Table 2, 729 classifications of anatomical positions can be provided. The apparatus 10 of the present invention may perform a classification based at least on the derived torso and limb positions to provide a classification of one of the 729 classifications.

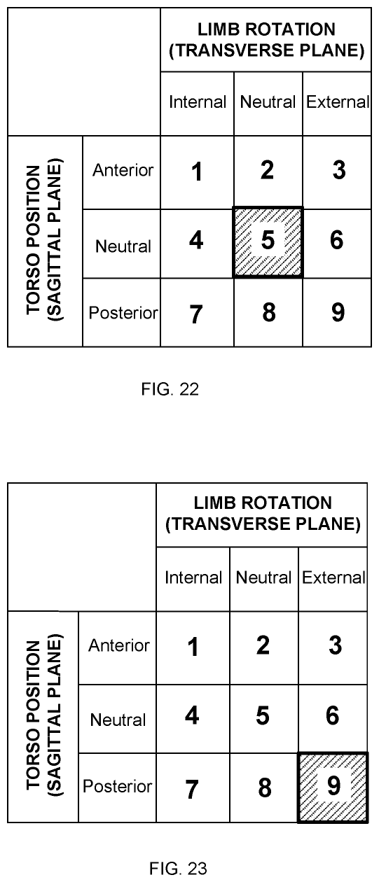

FIGS. 22 and 23 show simplified classification charts for torso and limb positions of a vertebral mammal 60 according to an embodiment of the present invention. The charts of FIGS. 22 and 23 simplify the classification algorithm 22 by only considering angular position of the torso 64 in the sagittal plane 50 and angular position of the limb 66 in the transverse plane 52. These positions often provide the most commonly occurring positional variations for vertebral mammals 60. When the classifier 34 classifies the derived positions using this simplified process, 9 possible classifications can be provided as shown in FIGS. 22 and 23. FIG. 22 shows an example of a classification of class 5 with neutral limb rotation and neutral torso position. This class signature may be indicative of high stability of a hip and/or shoulder joint of a mammal 60, with low risk for dislocation. In contrast, FIG. 23 shows an example of a classification of class 7 with internal limb rotation and anterior torso position. This class signature may be indicative of poor stability of a hip and/or shoulder joint of a mammal 60, with high risk for posterior dislocation of the joint.

FIG. 23 shows a flow chart for a simplified classification algorithm 22 for pelvic and femoral angular positions of a vertebral mammal 60 according to an embodiment of the present invention. In this embodiment, the classifier 34 of the algorithm 22 firstly classifies pelvic position in the sagittal plane 50 as one of anterior, neutral or posterior position or tilt. Secondly, the classifier 34 classifies the femoral position in the transverse plane 52 as one of internal, neutral or external rotation. There are 9 possible classes for classification, as similarly provided in the charts of FIGS. 22 and 23. For example, class 1 represents anterior pelvic tilt and internal femoral rotation, class 5 represents neutral pelvic tilt and neutral femoral rotation and class 9 represents posterior pelvic tilt and external femoral rotation.

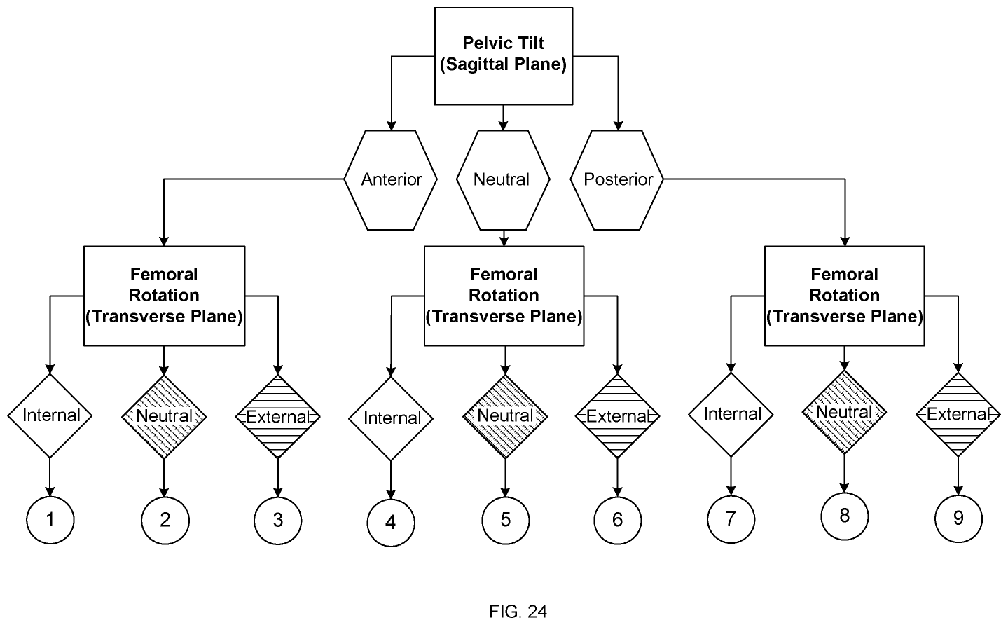

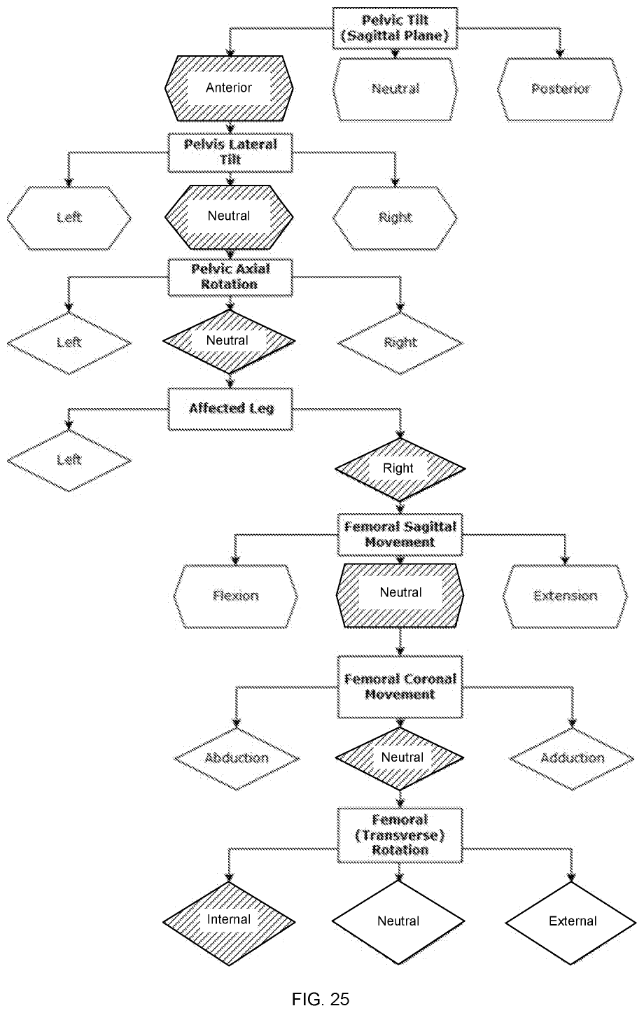

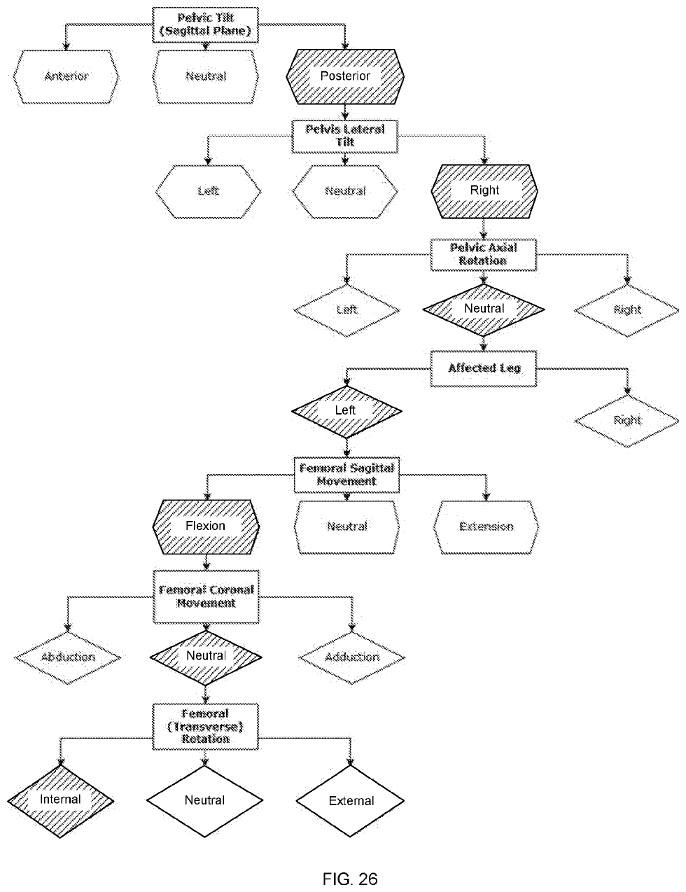

FIGS. 24 and 25 show flow charts for classification of pelvic and femoral positions of a vertebral mammal 60 according to an embodiment of the present invention, with respect to each of the sagittal, coronal and transverse planes 50-54 of the mammal's body 62. This involves identifying one of the 729 classifications from Table 3. In FIG. 25, the pelvic position is classified as anterior tilt, neutral lateral tilt and neutral rotation, and the femoral position is classified as neutral flexion, neutral lateral tilt and internal rotation. In FIG. 26, an alternative classification is illustrated. In this example, the pelvic position is classified as posterior tilt, right lateral tilt and neutral rotation, and the femoral position is classified as flexion, neutral lateral tilt and internal rotation.

An inventive apparatus 10 and method is proposed that can classify, based on derived angular positions of the torso and the limb, which class or group a subject or patient 60 belongs to. The different classification classes may guide a medical practitioner or surgeon in their clinical decisions regarding hip and/or shoulder replacement surgery or be able to classify the risk of injury based on a subject spending significant amount of time or force in one class or position. The different classes may be indicative of stability of the hip and/or shoulder joint. In particular, the ability to have an accurate assessment for position of the pelvis and the femur and/or tibia is important in the planning stage of a hip replacement. An initial assessment provides a baseline measure and may assist in the diagnosis and/or classification of hip and pelvic positions, guiding the surgeon's choice and alignment of the prosthesis. For example, this may guide a medical practitioner or surgeon as to which type of prosthesis to use and the expected alignment or desirable orientation of the native bone and prosthetic components in the joint.

The different classes may also be indicative of a range of motion of the torso and limb (in static and/or dynamic states) or asymmetries of the limbs and whether the range of motion is within normal limits or outside normal limits, and if so, by how much, through the comparison of torso and limb positions to reference data, such as from a normative database. This may be useful for evaluating a risk of injury, such as in the workplace, to the spine or upper limb.

Additionally, the ability to measure and monitor muscle activity of the muscles supporting the torso and limbs may assist in understanding if muscle activity is within normal levels, is similar right side versus left side of the subject's body or is acting with an aberrant pattern, that may indicate a pathological or abnormal state.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components.

It is to be understood that various modifications, additions and/or alternatives may be made to the parts previously described without departing from the ambit of the present invention as defined in the claims appended hereto.

It is also to be understood that the following claims are provided by way of example only, and are not intended to limit the scope of what may be claimed in any future application. Features may be added to or omitted from the claims at a later date so as to further define or re-define the invention or inventions.

### REFERENCES

[1] Dargel, J., Oppermann, J., Brüggemann, G. P. and Eysel, P., 2014. Dislocation following total hip replacement. Deutsches Ärzteblatt International, 111(51-52), p. 884.
[2] Lewinnek G E, Lewis J L, Tarr R, Compere C L, Zimmerman J R: Dislocations after total hip-replacement arthroplasties. J Bone Joint Surg 1978; 60: 217-20.
[3] Wines A P, McNicol D: Computed tomography measurement of the accuracy of component version in total hip arthroplasty. J Arthroplasty 2006; 21: 696-701.
[4] Gajdosik, R. et al. “Pelvic tilt. Intratester reliability of measuring the standing position and range of motion.” Physical therapy 65.2 (1985): 169; Azevedo, Daniel Camara, et a. “Reliability of sagittal pelvic position assessments in standing, sitting and during hip flexion using palpation meter.” Journal of bodywork and movement therapies 18.2 (2014): 210-214.
[5] Hopkins, Andrew R., et al. “The effects of glenoid component alignment variations on cement mantle stresses in total shoulder arthroplasty.” Journal of shoulder and elbow surgery 13.6 (2004): 668-675.

1. An apparatus for providing classification of position of a torso and a limb of a body of a vertebral mammal, said apparatus including:
a first sensor for measuring position of said torso relative to a first frame of reference and for providing first data indicative of said torso position;
a second sensor for measuring position of said limb relative to a second frame of reference and for providing second data indicative of said limb position;
a memory device adapted for storing said first and second data at least temporarily; and
a processor adapted for processing said first and second data to derive angular positions of said torso and said limb in at least one anatomical plane of said mammal's body, and to provide said classification based at least on said derived torso and limb positions.

2. The apparatus according to claim 1, wherein said processor is further adapted to execute a position algorithm for deriving an angular position of said limb relative to said torso in at least one anatomical plane of said mammal's body.

3. The apparatus according to claim 2, wherein said position algorithm is adapted to transform said second data from said second frame of reference relative to said first frame of reference to derive said angular position of said limb relative to said torso.

4. The apparatus according to claim 1, wherein said processor is further adapted to execute a classification algorithm for providing said classification based at least on said derived torso and limb positions.

5. The apparatus according to claim 4, wherein said processor is further adapted to receive reference data, and wherein said classification algorithm includes a comparator adapted to compare said derived torso and limb positions to said reference data.

6. The apparatus according to claim 5, wherein said reference data includes one or more threshold values or a range of values for said torso and limb positions based on a normative population of vertebral mammals.

7. The apparatus according to claim 5, wherein said classification algorithm includes a classifier adapted to one or both of:
classify said derived torso and limb positions based on said comparison by said comparator; and
at least classify said derived torso position in a sagittal plane of said mammal's body and classify said derived limb position in a transverse plane of said mammal's body.

8.-9. (canceled)

10. The apparatus according to claim 4, wherein said classification algorithm is further adapted to assign a class signature indicative of stability of a hip and/or a shoulder joint of said mammal based on said classification of said derived torso and limb positions.

11.-14. (canceled)

15. The apparatus according to claim 1, further including a third sensor for measuring muscle activity of said torso and/or limb of said mammal, and for providing third data indicative of said muscle activity.

16. The apparatus according to claim 1, wherein one of:
said torso includes a pelvis of said mammal and said limb includes a lower limb of said mammal; or
said torso includes a spine of said mammal and said limb includes an upper limb of said mammal.

17.-19. (canceled)

20. A method for providing classification of position of a torso and a limb of a body of a vertebral mammal, said method including:
measuring position of said torso relative to a first frame of reference using a first sensor;
measuring position of said limb relative to a second frame of reference using a second sensor;
providing first data indicative of said torso position and second data indicative of said limb position;
storing said first and second data at least temporarily; and
processing said first and second data to derive angular positions of said torso and said limb in at least one anatomical plane of said mammal's body, and to provide said classification based at least on said derived torso and limb positions.

21. The method according to claim 20, wherein said processing further includes performing a position algorithm for deriving an angular position of said limb relative to said torso in at least one anatomical plane of said mammal's body.

22. The method according to claim 21, wherein said performing said position algorithm includes transforming said second data from said second frame of reference relative to said first frame of reference to derive said angular position of said limb relative to said torso.

23. The method according to claim 20, wherein said processing further includes performing a classification algorithm for providing said classification based at least on said derived torso and limb positions.

24. The method according to claim 23, further including receiving reference data, and wherein performing said classification algorithm includes comparing, using a comparator of said classification algorithm, said derived torso and limb positions to said reference data.

25. The method according to claim 24, wherein said reference data includes one or more threshold values or a range of values for said torso and limb positions based on a normative population of vertebral mammals.

26. The method according to claim 24, wherein said performing said classification algorithm further includes one or both of:
classifying, using a classifier of said classification algorithm, said derived torso and limb positions based on said comparison by said comparator; and
at least classifying said derived torso position in a sagittal plane of said mammal's body and classifying said derived limb position in a transverse plane of said mammal's body.

27.-28. (canceled)

29. The method according to claim 23, wherein said performing said classification algorithm further includes assigning a class signature indicative of stability of a hip and/or a shoulder joint of said mammal based on said classification of said derived torso and limb positions.

30.-33. (canceled)

34. The method according to claim 20, further including measuring muscle activity of said torso and/or limb of said mammal using a third sensor, and providing third data indicative of said muscle activity.

35. The method according to claim 20, wherein one of:
said torso includes a pelvis of said mammal and said limb includes a lower limb of said mammal;
said torso includes a spine of said mammal and said limb includes an upper limb of said mammal.

36.-38. (canceled)
